(19) United States
(12) Patent Application Publication
(10) Pub. No.: US 2023/0030773 A1
(43) Pub. Date: Feb. 2, 2023

(54) USE OF POLYVINYL ALCOHOL FOR CELL CULTURE OF IMMUNE CELLS

(71) Applicant: The Board of Trustees of the Leland Stanford Junior University, Stanford, CA (US)

(72) Inventors: Adam C. Wilkinson, Redwood City, CA (US); Hiromitsu Nakauchi, Woodside, CA (US); Yamazaki Satoshi, Tokyo (JP); Kyle M. Loh, Stanford, CA (US); Toshinobu Nishimura, Redwood City, CA (US)

(21) Appl. No.: 17/783,473

(22) PCT Filed: Dec. 15, 2020
(86) PCT No.: PCT/US2020/065103

§ 371 Date: Jun. 8, 2022

Related U.S. Application Data
(60) Provisional application No. 62/948,630, filed on Dec. 16, 2019.

(51) Int. Cl. C12N 5/0783; C12N 15/09; C07K 14/725

(52) U.S. Cl. CPC C12N 5/0636; C12N 15/09; C07K 14/7051; C12N 5/0037

(57) ABSTRACT

Serum albumin-free media comprising polyvinyl alcohol (PVA) and methods of culturing immune cells in such media are disclosed. The PVA is used as a replacement for fetal bovine serum, bovine serum albumin, and recombinant serum albumin in media. Advantages of using PVA include that it is a chemically-defined reagent that is available at high-purity with minimal batch-to-batch variability.

### STATEMENT REGARDING FEDERALLY SPONSORED RESEARCH OR DEVELOPMENT

This invention was made with Government support under contract OD024558 awarded by the National Institutes of Health. The Government has certain rights in the invention.

### BACKGROUND

Serum albumin has long been an essential supplement for ex vivo cultures of hematopoietic cells and lymphocytes (Francis et al. (2010) Cytotechnology 62(1):1-16). However, serum albumin supplements represent a major source of biological contaminants and often display significant batch-to-batch variability, causing issues in experimental reproducibility and time-consuming batch-testing (leyasu et al. (2017) Stem Cell Reports 8(3):500-508). Progressive attempts to limit these contaminants have involved moving from the use of serum such as fetal bovine serum (FBS) or human serum to purified serum albumin (e.g. bovine serum albumin fraction V) and/or recombinantly-derived human serum albumin² (HSA). Additionally, serum albumin supplements represent a major cost in cell culture, particularly where Good Manufacturing Practice (GMP)-grade reagents are required.

There remains a need to find better supplements that can replace serum albumin for cell culture.

### SUMMARY

Serum albumin-free media comprising polyvinyl alcohol (PVA) and methods of culturing immune cells in such media are disclosed. The PVA is used as a replacement for fetal bovine serum, bovine serum albumin, and recombinant serum albumin in media. Advantages of using PVA include that it is a chemically-defined reagent that is available at high-purity with minimal batch-to-batch variability.

In one aspect, a method of culturing immune cells is provided, the method comprising culturing the immune cells in a serum albumin-free medium comprising polyvinyl alcohol (PVA).

In certain embodiments, the immune cells are lymphocytes. In some embodiments, the lymphocytes are T cells. In some embodiments, the T cells are chimeric antigen receptor (CAR) T cells.

In certain embodiments, the serum albumin-free medium further comprises one or more cytokines or growth factors. Exemplary cytokines and growth factors include, without limitation, interleukin 1 (IL-1), interleukin 2 (IL-2), interleukin 3 (IL-3), interleukin 4 (IL-4), interleukin 6 (IL-6), interleukin 7 (IL-7), interleukin 9 (IL-9), interleukin 10 (IL-10), interleukin 10 (IL-12), interleukin 13 (IL-13), interleukin 15 (IL-15), interleukin 18 (IL-18), interleukin 21 (IL-21), interleukin 23 (IL-23), tumor necrosis factor alpha (TNFα), tumor necrosis factor beta (TNFβ), interferon gamma (IFN-γ), macrophage colony-stimulating factor (M-CSF), granulocyte-macrophage colony-stimulating factor (GM-CSF), thrombopoietin (TPO), stem cell factor (SCF), and FMS-like tyrosine kinase 3 ligand (FLT3L).

In certain embodiments, the serum albumin-free medium further comprises insulin, transferrin, selenium, or ethanolamine, or a combination thereof.

In certain embodiments, the serum albumin-free medium further comprises an antibiotic. Exemplary antibiotics that can be used in cell culture include, without limitation, penicillin, streptomycin, penicillin-streptomycin (PenStrep), gentamicin, amphotericin, and the like.

In certain embodiments, the PVA is at a concentration of about 0.1 mg/ml to about 10 mg/ml in the serum albumin-free medium, including any concentration within this range such as 0.1, 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml. In one embodiment, the PVA is at a concentration of about 1 mg/ml in the serum albumin-free medium.

In certain embodiments, the culturing is performed in a suspension culture or an adherent culture.

In another aspect, a composition is provided comprising expanded immune cells cultured in a serum albumin-free media comprising polyvinyl alcohol (PVA) as described herein.

In certain embodiments, the composition further comprises a pharmaceutically acceptable excipient.

In another aspect, a method of cellular therapy is provided comprising administering a composition comprising expanded immune cells cultured in a serum albumin-free media comprising polyvinyl alcohol (PVA), as described herein, to a subject.

In certain embodiments, the immune cells are autologous or allogeneic.

In another aspect, a method of adoptive cellular therapy with immune cells is provided, the method comprising: a) obtaining a biological sample comprising immune cells from a subject; b) isolating the immune cells of interest from the biological sample; c) expanding the immune cells in a serum albumin-free cell culture medium comprising polyvinyl alcohol; and d) administering a therapeutically effective amount of the expanded immune cells of interest to the subject.

In certain embodiments, the biological sample is blood.

In certain embodiments, the immune cells are lymphocytes. In some embodiments, the lymphocytes are T cells.

In certain embodiments, the method further comprises genetically modifying the T cells to produce a chimeric antigen receptor (CAR) prior to administering said T cells to the subject. For example, a T cell can be genetically modified to produce a CAR that specifically binds to a cancer epitope for treating a subject who has cancer. In some embodiments, the method further comprises expanding the CAR T cells in the serum albumin-free cell culture medium comprising polyvinyl alcohol prior to administering said CAR T cells to the subject.

In another aspect, a serum albumin-free cell culture medium comprising polyvinyl alcohol and one or more cytokines is provided. Exemplary cytokines include, without limitation, interleukin 1 (IL-1), interleukin 2 (IL-2), interleukin 3 (IL-3), interleukin 4 (IL-4), interleukin 6 (IL-6), interleukin 7 (IL-7), interleukin 9 (IL-9), interleukin 10 (IL-10), interleukin 10 (IL-12), interleukin 13 (IL-13), interleukin 15 (IL-15), interleukin 18 (IL-18), interleukin 21 (IL-21), interleukin 23 (IL-23), tumor necrosis factor alpha (TNFα), tumor necrosis factor beta (TNFβ), interferon gamma (IFN-γ), macrophage colony-stimulating factor (M-CSF), granulocyte-macrophage colony-stimulating factor (GM-CSF), thrombopoietin (TPO), stem cell factor (SCF), and FMS-like tyrosine kinase 3 ligand (FLT3L).

In certain embodiments, the medium further comprises insulin, transferrin, selenium, or ethanolamine, or a combination thereof.

In another aspect, a kit comprising a serum albumin-free medium comprising PVA, described herein, is provided. The kit may further comprise instructions for culturing immune cells in the serum albumin-free medium comprising PVA.

In another aspect, a culture system comprising a container containing a serum albumin-free medium comprising PVA, described herein, is provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1E show that poly-vinyl alcohol can replace serum albumin in leukemic cell cultures. (FIG. 1A) ELISA assay for mouse TPO stability in RPM1-based culture media after 48 hours (initiated at 10 ng/ml). Mean±S.D of technical duplicates from biological replicates. (FIG. 1B) Schematic summary of TPO-dependent mouse 32D/MPL cells grown in various RPMI-based medias supplemented 10 ng/ml TPO. (FIG. 1C) Total numbers of 32D/MPL cells after a 14-day culture in the RMPI-based medias described in (FIG. 1B) with TPO at 10 ng/ml. Dotted line indicates starting cell concentration. Mean±S.D of four independent cultures. (FIG. 1D) Total numbers of primary human AML cells (two AML samples from the Stanford Hematology Biobank) after a 14-day culture in MEMa-based medias, with indicated supplements and cytokines (TPO, SCF, FLT3L, IL-6, IL-3, GM-CSF, and G-CSF; all 20 ng/ml). Dotted line indicates starting cell concentration. Mean±S.D of four independent cultures. (FIG. 1E) Total numbers of human K-562 cells over a 14-day culture in the RMPI-based medias with indicated supplements. Mean of four independent cultures. In all panels, statistical analysis was performed by one-way ANOVA (analysis measured against the PVA+ITSX condition with significance indicated by: *, p<0.05, **, p<0.01, ***, p>0.001; ****, p>0.0001; n.s, non-significant.

FIGS. 2A-2D show that poly-vinyl alcohol can replace serum albumin in T lymphocyte cell cultures. (FIG. 2A) ELISA assay for human IL-15 stability in culture media after 48 hours (initiated at 10 ng/ml). Mean±S.D of technical duplicates from biological replicates. (FIG. 2B) Total number of primary T cells after an 11-day culture in RPMI-based medias with the indicated supplements and cytokines (IL-15 and IL-7; 10 ng/ml each), following stimulation with anti-CD3/CD28 dynabeads. Mean±S.D of triplicated experiments for two healthy donors. (FIG. 2C) Schematic of CAR-T cell production by primary T cell collection and stimulation with anti-CD3/CD28 dynabeads at day 0, lentiviral transduction at days 2 and 3, and expansion until day 10, all in RMPI+PVA+ITSX and RPMI+Serum+ITSX. At day 10, transduced mCherry+anti-CD19-CAR T cells were used in a killing assay using CD19+NALM6 target cells at a ratio of 5:1 (CAR-T cells:target cells). (FIG. 2D) Killing activity of anti-CD19 CAR-T cells targeting CD19-expressing NALM6 cells, described in (FIG. 2C). Mean±S.D of technical triplicates for two separate donors. Representative of two biological replicates. Statistical analysis was performed by one-way ANOVA (FIGS. 2A-2B) or two-way ANOVA (FIG. 2D) analysis measured against the PVA+ITSX condition with significance indicated by: *, p<0.05, **, p<0.01, ***, p>0.001; ****, p>0.0001; n.s, non-significant.

### DETAILED DESCRIPTION

Serum albumin-free media comprising polyvinyl alcohol (PVA) and methods of culturing immune cells in such media are disclosed. The PVA is used as a replacement for fetal bovine serum, bovine serum albumin, and recombinant serum albumin in media. Advantages of using PVA include that it is a chemically-defined reagent that is available at high-purity with minimal batch-to-batch variability.

Before the serum albumin-free media comprising PVA and its use in culturing immune cells are further described, it is to be understood that this invention is not limited to a particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the T cells” includes reference to one or more T cells and equivalents thereof, e.g., T lymphocytes, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

As used herein, the term “affinity” refers to the equilibrium constant for the reversible binding of two agents and is expressed as a dissociation constant (Kd). Affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more, than the affinity of an antibody for unrelated amino acid sequences. Affinity of an antibody to a target protein can be, for example, from about 100 nanomolar (nM) to about 0.1 nM, from about 100 nM to about 1 picomolar (pM), or from about 100 nM to about 1 femtomolar (fM) or more. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges. Non-specific binding would refer to binding with an affinity of less than about 10⁻⁷ M, e.g., binding with an affinity of 10⁻⁶ M, 10⁻⁵ M, 10⁻⁴ M, etc.

An “isolated” polypeptide is one that has been identified and separated and/or recovered from a component of its natural environment. Contaminant components of its natural environment are materials that would interfere with diagnostic or therapeutic uses for the polypeptide, and may include enzymes, hormones, and other proteinaceous or nonproteinaceous solutes. In some embodiments, the polypeptide will be purified (1) to greater than 90%, greater than 95%, or greater than 98%, by weight of antibody as determined by the Lowry method, for example, more than 99% by weight, (2) to a degree sufficient to obtain at least 15 residues of N-terminal or internal amino acid sequence by use of a spinning cup sequenator, or (3) to homogeneity by sodium dodecyl sulfate-polyacrylamide gel electrophoresis (SDS-PAGE) under reducing or nonreducing conditions using Coomassie blue or silver stain. Isolated polypeptide includes the polypeptide in situ within recombinant cells since at least one component of the polypeptide's natural environment will not be present. In some instances, isolated polypeptide will be prepared by at least one purification step.

By “container” is meant a glass, plastic, or metal vessel that can provide an aseptic environment for culturing cells.

As used herein, the term “immune cells” generally includes white blood cells (leukocytes) which are derived from hematopoietic stem cells (HSC) produced in the bone marrow. “Immune cells” include, without limitation, lymphocytes (e.g., T cells, B cells, natural killer (NK) cells) and myeloid-derived cells (neutrophils, eosinophils, basophils, monocytes, macrophages, and dendritic cells).

The term “T cell” includes all types of immune cells expressing CD3 including T-helper cells (CD4⁺ cells), cytotoxic T-cells (CD8⁺ cells), T-regulatory cells (Treg) and gamma-delta T cells.

A “cytotoxic cell” includes CD8⁺ T cells, natural-killer (NK) cells, and neutrophils, which cells are capable of mediating cytotoxicity responses.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

The term “biological sample” encompasses a clinical sample. The types of “biological samples” include, but are not limited to: a bodily fluid, tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like.

The terms “obtained” or “obtaining” as used herein can also include the physical extraction or isolation of a biological sample (e.g., comprising immune cells) from a subject. Accordingly, a biological sample comprising immune cells can be isolated from a subject (and thus “obtained”) by the same person or same entity that subsequently isolates immune cells from the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample. Methods and protocols for isolating various biological samples (e.g., a blood sample, a biopsy sample, an aspirate, etc.) will be known to one of ordinary skill in the art and any convenient method may be used to isolate a biological sample.

“Substantially purified” generally refers to isolation of a component of a sample (e.g., cell or substance), such that the component comprises the majority percent of the sample in which it resides. Typically in a sample, a substantially purified component comprises at least 70%, preferably at least 80%-85%, more preferably at least 90-99% of the sample.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted as well as those in which prevention is desired (e.g., those with increased susceptibility to an autoimmune disease, etc.)

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly salts containing pharmaceutically acceptable cations include, but are not limited to, sodium, potassium, calcium, aluminum, lithium, and ammonium (including substituted ammonium).

An “effective amount” of a composition comprising immune cells is an amount sufficient to safely effect beneficial or desired results. An effective amount can be administered in one or more administrations, applications, or dosages.

The term “unit dosage form,” as used herein, refers to physically discrete units suitable as unitary dosages for human and animal subjects, each unit containing a predetermined quantity of the agents calculated in an amount sufficient to produce the desired effect in association with a pharmaceutically acceptable diluent, carrier or vehicle. The specifications for the unit dosage forms for use in the present invention depend on the particular compound employed and the effect to be achieved, the pharmacodynamics associated with each compound in the host, and the like.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

“Operably linked” refers to an arrangement of elements wherein the components so described are configured so as to perform their usual function. Thus, a given promoter operably linked to a coding sequence is capable of effecting the expression of the coding sequence when the proper enzymes are present. Expression is meant to include the transcription of mRNA from a DNA or RNA template and can further include translation of a protein from an mRNA template. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

A “vector” is capable of transferring nucleic acid sequences to target cells (e.g., viral vectors, non-viral vectors, particulate carriers, and liposomes). Typically, “vector construct,” “expression vector, ” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant” refers to biologically active derivatives of the reference molecule that retain desired activity. In general, the term “variant” refers to molecules having a native sequence and structure with one or more additions, substitutions (generally conservative in nature) and/or deletions, relative to the native molecule, so long as the modifications do not destroy biological activity and which are “substantially homologous” to the reference molecule. In general, the sequences of such variants will have a high degree of sequence homology to the reference sequence, e.g., sequence homology of more than 50%, generally more than 60%-70%, even more particularly 80%-85% or more, such as at least 90%-95% or more, when the two sequences are aligned.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

By “vertebrate subject” is meant any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered.

### Serum Albumin-Free Media Comprising Polyvinyl Alcohol

A serum albumin-free media comprising PVA is provided for culturing immune cells.

The PVA is used as a replacement for fetal bovine serum, bovine serum albumin, and recombinant serum albumin in media. Advantages of using PVA include that it is a chemically-defined reagent that is available at high-purity with minimal batch-to-batch variability.

The serum-free culture media to which the PVA is added will generally include a buffer, protein, inorganic salts, trace elements, vitamins, lipids, and carbohydrates. In some embodiments, the PVA concentration in the serum albumin-free media ranges from about 0.1 mg/ml to 10 mg/ml, including any concentration in this range such as 0.1, 0.2, 0.4, 0.6, 0.8, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10 mg/ml. In one embodiment, the PVA is at a concentration of about 1 mg/ml in the serum albumin-free medium.

Any type of immune cell may be cultured in the serum albumin-free media comprising PVA. For example, immune cells, including, without limitation, monocytes, T cells such as CD8⁺ T cells (e.g., cytotoxic T cells) and CD4⁺ T cells (e.g., Th1, Th2, Th17, and Treg), NK cells, B cells, neutrophils, eosinophils, and basophils may be cultured in the serum albumin-free media comprising PVA.

The cells cultured in the serum albumin-free media comprising PVA may include immune cells isolated from a biological sample such as a bodily fluid (e.g., blood) or tissue obtained from a subject. For example, immune cells can be obtained from blood, peripheral blood mononuclear cells (PBMCs), bone marrow, the lymphatic system, or inflamed tissue in which immune cells have infiltrated. An immune cell can be a primary cell obtained directly from a subject. Alternatively, the immune cell may be from a cell line or a cell derived from the culture and expansion of a cell obtained from a subject.

In some embodiments, antigen-specific immune cells (e.g., antigen-specific T cells or B cells) are expanded in the serum albumin-free media comprising PVA. Such cells can be obtained by eliciting an immune response to an antigen of interest. Lymphocytes may be contacted with an antigen of interest (T cells in the presence of an antigen presenting cell) in vivo, ex vivo, or in vitro. For example, the antigen of interest can be administered to a subject to elicit an immune response followed by collection of a biological sample from the subject comprising immune cells recognizing the antigen of interest. The biological sample may be any sample containing lymphocytes (e.g., B cells or T cells) specific for the antigen of interest, such as a blood sample, a sample of peripheral blood mononuclear cells (PBMCs), or inflamed tissue in which antigen-specific lymphocytes have infiltrated. Alternatively, a biological sample comprising lymphocytes can be collected from a subject and treated with an antigen of interest (T cells in the presence of an antigen-presenting cell) ex vivo or in vitro.

Examples of suitable antigen presenting cells that can present an antigen of interest to T lymphocytes include dendritic cells, macrophages, and activated B cells. Alternately, artificial antigen presenting cells may be used, such as soluble major histocompatibility complex (MHC)-multimers or cellular or acellular artificial antigen presenting cells. MHC-multimers typically range in size from dimers to octamers (tetramers commonly used) and can be used to display class 1 or class 2 MHC (Hadrup et al. (2009) Nature Methods 6:520-526, Nepom et al. (2003) Antigen 106:1-4, Bakker et al. (2005) Current Opinion in Immunology 17:428-433). Cellular artificial antigen presenting cells may include cells that have been genetically modified to express T-cell co-stimulatory molecules, MHC alleles and/or cytokines. For example, artificial antigen presenting cells have been generated from fibroblasts modified to express HLA molecules, the co-stimulatory signal, B7.1, and the cell adhesion molecules, ICAM-1 and LFA-3 (Latouche et al. (2000) Nature Biotechnology. 18 (4):405-409). Acellular antigen presenting cells comprise biocompatible particles such as microparticles or nanoparticles that carry T cell activating proteins on their surface (Sunshine et al. (2014) Biomaterials. 35 (1): 269-277), Perica et al. (2014) Nanomedicine: Nanotechnology, Biology and Medicine. 10 (1):119-129). For a review of artificial antigen presenting cells, see, e.g., Oelke et al. (2004) Clin. Immunol. 110(3):243-251, Wang et al. (2017) Theranostics 7(14):3504-3516, Butler et al. (2014) Immunol Rev. 257(1):191-209, Eggermont et al. (2014) Trends Biotechnol. 32(9):4564-4565, Sunshine et al. (2013) Nanomedicine (Lond) 8(7):1173-1189, and Rhodes et al. (2018) Mol. Immunol. 98:13-18; herein incorporated by reference.

Typically, the antigen of interest is at a concentration ranging from about 10 μg/ml to about 40 μg/ml in the biological sample. The antigen of interest may be pre-incubated with the antigen presenting cells for periods ranging from 1 to 18 hours prior to stimulation of the CD4⁺ T lymphocytes. Culture media may be supplemented with interleukin 2 (IL-2) and interleukin 15 (IL-15) during intervals between stimulations to induce amino acid uptake and protein synthesis in antigen-activated T cells to promote growth and proliferation of antigen-specific T lymphocytes. The antigen-specific immune cells can be subsequently expanded in culture in the serum albumin-free media comprising PVA.

Different types of immune cells may have different medium requirements and need different types of media supplements to support cell growth, proliferation, differentiation, and survival. In some embodiments, the culture media is supplemented with one or more factors, such as cytokines, growth factors, antibiotics, or other media supplements, and the like. Exemplary cytokines and growth factors include, without limitation, interleukin 1 (IL-1), interleukin 2 (IL-2), interleukin 3 (IL-3), interleukin 4 (IL-4), interleukin 6 (IL-6), interleukin 7 (IL-7), interleukin 9 (IL-9), interleukin 10 (IL-10), interleukin 10 (IL-12), interleukin 13 (IL-13), interleukin 15 (IL-15), interleukin 18 (IL-18), interleukin 21 (IL-21), interleukin 23 (IL-23), tumor necrosis factor alpha (TNFα), tumor necrosis factor beta (TNFβ), interferon gamma (IFN-γ), macrophage colony-stimulating factor (M-CSF), granulocyte-macrophage colony-stimulating factor (GM-CSF), thrombopoietin (TPO), stem cell factor (SCF), and FMS-like tyrosine kinase 3 ligand (FLT3L). In some embodiments, media supplements including cytokines and growth factors are chosen to support differentiation and/or expansion of cells and will depend on the specific types of immune cells being cultured. For example, one or more cytokines or growth factors may be selected from specific subsets of cytokines known to have activity on a specific cell type, e.g., for culture of CD8⁺ T cells, including cytotoxic T cells (e.g., IL-2, IL-15, and IL-7); CD4⁺ T cells, including Th1 cells (e.g., IL-2, IFN-γ, IL-12), Th2 cells (e.g., IL-2, IL-4), Th17 cells (e.g., TGF-β1, IL-6, IL-21, IL-1β, TGF-β3, and IL-23), and Treg cells (e.g., TGF-β1); macrophages, including M1 macrophages (e.g., M-CSF, GM-CSF, IFN-γ, and TNF-α); M2a macrophages (e.g., M-CSF, IL-13, and IL-4); M2b macrophages (e.g., M-CSF and IL-1β); M2c macrophages (e.g., M-CSF, IL-10, and TGF-β1); dendritic cells (e.g., GM-CSF and IL-4); natural killer (NK) cells (e.g., IL-2, IL-12, IL-18, and IL-15); and B cells (e.g., IL-4 and IL-7). In some embodiments, the concentration of a cytokine in the media ranges from about 1.0 ng/ml to 30 ng/ml, including any concentration in this range such as 1, 2, 3, 4, 5, 6, 7, 8, 9, 10, 11, 12, 13, 14, 15, 16, 17, 18, 19, 20, 21, 22, 23 , 24, 25, 26, 27, 28, 29, or 30 ng/ml.

In some embodiments, one or more cytokine T-cell growth factors including, without limitation, IL-2, IL-7, IL-9, and IL-15 are used to promote expansion of T cells. IL-2 is commonly used for expansion of T cells, but other interleukins may be used for specific subsets of T cells. In some embodiments, T cells are stimulated by an anti-CD3 antibody and IL-2 during expansion in the serum albumin-free media comprising PVA.

In addition, T cells may be activated in culture by antigen presentation. For example, an antigen presenting cell such as a dendritic cell, macrophage, B cell, or microglial cell may be added to the culture. Alternatively, an artificial antigen presenting cell may be used such as, but not limited to, a major histocompatibility complex (MHC) multimer (e.g., dimer, tetramer, pentamer, octamer, dextramer), a cellular artificial antigen presenting cell (e.g., fibroblasts or other cells genetically modified to express MHC and other T cell stimulating proteins), or an acellular antigen presenting cell (e.g., biocompatible particle such as a microparticle or nanoparticle carrying T cell stimulating proteins).

In some embodiments, an antibiotic is added to the culture media to prevent bacterial contamination. Exemplary antibiotics that can be used in cell culture include, without limitation, penicillin, streptomycin, penicillin-streptomycin (PenStrep), gentamicin, amphotericin, and the like. The use of such antibiotics may be undesirable if they are found to impair cell growth and/or differentiation of the cells. Alternatively, the use of good aseptic laboratory practice may make the use of antibiotics unnecessary. Aseptic techniques include maintaining a sterile work area, sterile reagents and media, and sterile handling of culture manipulations.

Other supplements that may be added to the serum albumin-free media comprising PVA include, without limitation, insulin, transferrin, selenium, or ethanolamine, or a combination thereof.

A culture system utilizing the serum albumin-free media comprising PVA may be used for short term culture, long term culture, growth, proliferation and expansion of immune cells. For example, the serum albumin-free media comprising PVA may be used for long term expansion or growth of immune cells for more than 10 days, more than 30 days, more than 60 days, more than 100 days, more than 150 days, or longer. Alternatively, the serum albumin-free media comprising PVA may be used for short term expansion or growth of immune cells for less than 10 days, such as 10 days, 9 days, 8 days, 7 days, 6 days, 5 days, 4 days, 3 days, 2 days, or 1 day.

Cellular Therapy with T cells

T cell therapy can be performed with T cells expanded in the serum albumin-free media comprising PVA. In some embodiments, the T cells chosen for expansion and T cell therapy express a T cell receptor specific for a particular antigen of interest. The use of antigen-specific T cells avoids the risk of non-specific T cell responses. In certain embodiments, the antigen specificity of a T cell is controlled by introducing an exogenous T cell receptor having specificity for an antigen of interest into a T cell. For example, T cells can be transduced with a viral vector encoding a natural T cell receptor or an artificial T cell receptor such as a chimeric antigen receptor (CAR) having the desired specificity for an antigen of interest. CARs are artificial T cell receptors that typically comprise a single-chain variable fragment (scFv), which provides antigen specificity, fused to a T cell co-stimulatory domain and an activation domain. CARs can recognize epitopes of a target antigen of interest expressed on a cell surface (e.g., B cells or antigen presenting cells), but are not MHC-restricted. In T cells, designed with exogenous T cell receptors (e.g., natural or artificial) specific for an antigen of interest, the gene for the endogenous T cell receptor may be inactivated or deleted. T cells can be expanded in a serum albumin-free media comprising PVA before or after genetically modifying them. For a review of engineering antigen-specific Tregs with exogenous T cell receptors and CAR receptors, see, e.g., Dawson et al. (2017) Transl. Res. 187:53-58, Boardman et al. (2016) Biochem. Soc. Trans. 44(2):342-8, Adair et al. (2017) Front. Immunol. 8:1117, Zhang et al. (2018) Front. Immunol. 2018; 9: 2359, Tsang et al. (2008) J. Clin. Invest. 118(11):3619-28, Kim et al. (2015) Blood 125(7):1107-15, Brusko et al. (2010) PLoS One 5(7):e11726, Wright et al. (2009) Proc. Natl. Acad. Sci. U.S.A. 106(45):19078-83, Schubert et al. (2018) Int. J. Cancer. 142(9):1738-1747; herein incorporated by reference.

### Kits

Any of the compositions described herein may be included in a kit. For example, a kit may comprise a serum albumin-free media, PVA (e.g., in the serum albumin-free media or separate), and optionally one or more other factors, such as cytokines (e.g., TPO, SCF, FLT3L, IL-2, IL-3, IL-6, IL-7, IL-15, TNFα, IFN-γ, and GM-CSF), growth factors, antibiotics, or other media supplements, and the like. In addition, a kit may further comprise immune cells (e.g., in a serum albumin-free media comprising PVA or separate). In some embodiments, the kit further comprises a vector for genetically modifying immune cells. For example, the kit may comprise a vector for genetically modifying a T cell to produce an exogenous T cell receptor or chimeric antigen receptor. Additionally, the kit may include transfection agents, buffers, tissue culture plates or flasks, and the like.

Such kits generally will comprise, in suitable means, distinct containers for each individual reagent or solution. The kit may comprise one or more containers holding the serum albumin-free media comprising PVA, immune cells, and/or a vector, and/or transfection agents, and other agents. Suitable containers for the compositions include, for example, bottles, vials, syringes, and test tubes. Containers can be formed from a variety of materials, including glass or plastic. A container may have a sterile access port (for example, the container may be a vial having a stopper pierceable by a hypodermic injection needle).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, flash drive, and the like, on which the information has been recorded. Yet another form of these instructions that may be present is a website address which may be used via the internet to access the information at a removed site.

It will be apparent to one of ordinary skill in the art that various changes and modifications can be made without departing from the spirit or scope of the invention.

### Experimental

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention.

### EXAMPLE 1

### Use of Polyvinyl Alcohol for CAR T Cell Expansion

We recently identified polyvinyl alcohol (PVA) in combination with insulin-selenium-transferrin-ethanolamine (ITSX) as a serum replacement for ex vivo hematopoietic stem cell (HSC) culture³. In comparison to serum albumin media supplements, PVA represents an inexpensive, chemically-defined, and GMP-compatible alternative. Here, we demonstrate that PVA can also be used as a serum albumin replacement for in vitro culture of leukemia cells and T lymphocytes, including Chimeric Antigen Receptor (CAR) T cells. Adaptive immunotherapies such as CAR T cell therapies have become an exciting new therapeutic approach to treat and cure various cancers⁴⁻⁶. However, the large-scale ex vivo expansion of T lymphocytes⁷ for these therapies currently rely on expensive pre-screened human serum. Our results suggest that PVA is a chemically-defined alternative to serum albumin for CAT T cell expansion, which may offer advantages in terms of cost, safety, and reproducibility.

### Materials and Methods:

### Cell Line Cultures

Mouse 32D/MPL (previously generated by the laboratory⁸) and human K562 cells (purchased from ATCC) were cultured in RPM11640 media (Gibco) supplemented with 10% (v/v) fetal bovine serum (FBS; Sigma), 1% insulin-transferrin-selenium-ethanolamine (ITSX; Gibco), 1 mg/ml recombinant human serum albumin (HSA; Albumin Biosciences), and/or 1 mg/ml polyvinyl alcohol (PVA; Sigma) at 37° C. with 5% CO₂. 32D/MPL cells were supplemented with 10 ng/ml recombinant mouse TPO (R&D Systems or Peprotech). Both cell lines tested negative for mycoplasma using the MycoAlert Plus Mycoplasma Detection Kit (Lonza), following manufacturer instructions. Cell counting was performed using the Chemometec NC-3000 automated cell counter.

### Primary AML Cell Cultures

Primary human AML samples were collected and stored by the Stanford Hematology Tissue Bank, following informed patient consent. according to the Administrative Panel on Human Subjects Research Institutional Review Board (IRB)-approved protocols (Stanford IRB no. 18329, no. 6453, and no. 5637). Primary AML cells were cultured in MEMa (containing 1% Penicillin-Streptomycin-Glutamine; Gibco) supplemented with 10% FBS, 1% ITSX, and/or 1 mg/ml PVA at 37° C. with 5% CO₂. The following cytokines were added to the culture (as described previously⁹): human TPO, human SCF, human FLT3L, human IL-3, human IL-6, human GM-CSF, and human G-CSF (all at 20 ng/ml).

### Primary T Cell Cultures

Peripheral blood mononuclear cells (PBMCs) from de-identified healthy donors were purchased from the Stanford Blood Center. CD3+ T cells were purified and stimulated with anti-CD3/CD28 dynabeads, then cultured in RPM11640 media (Gibco) supplemented with 10 ng/ml IL-15, 10 ng/ml IL-7, 5% (v/v) human serum, 1% insulin-transferrin-selenium-ethanolamine (ITSX; Gibco), and/or 1 mg/ml PVA at 37° C. with 5% CO₂.

### CAR T Cell Assays

CD3+ T cells were purified and stimulated as above, before lentiviral transduction on day 2 and 3 with anti-CD19-CAR lentivirus (lentivirus plasmid was provided by Dr. Crystal Mackall with slight modification). Lentiviral infection was conducted with Retronectin coating (Clontech) with spinfection (1,000 g for 1 hour at 32° C.) at MOI=10. After an additional 7-day expansion, in vitro killing activity was determined by bioluminescent-mediated viability measurement¹⁰ with firefly luciferase (FLuc)-transduced NALM6 cells.

### ELISA Assays

Mouse TPO and human IL-15 ELISA Kits were purchased from R&D Systems (MTP00 and D1500) and performed according manufacturer instructions.

### Statistical Analysis

One-way and two-way ANOVA tests were performed as indicated in the figures using Prism 7 software.

### Results and Discussion:

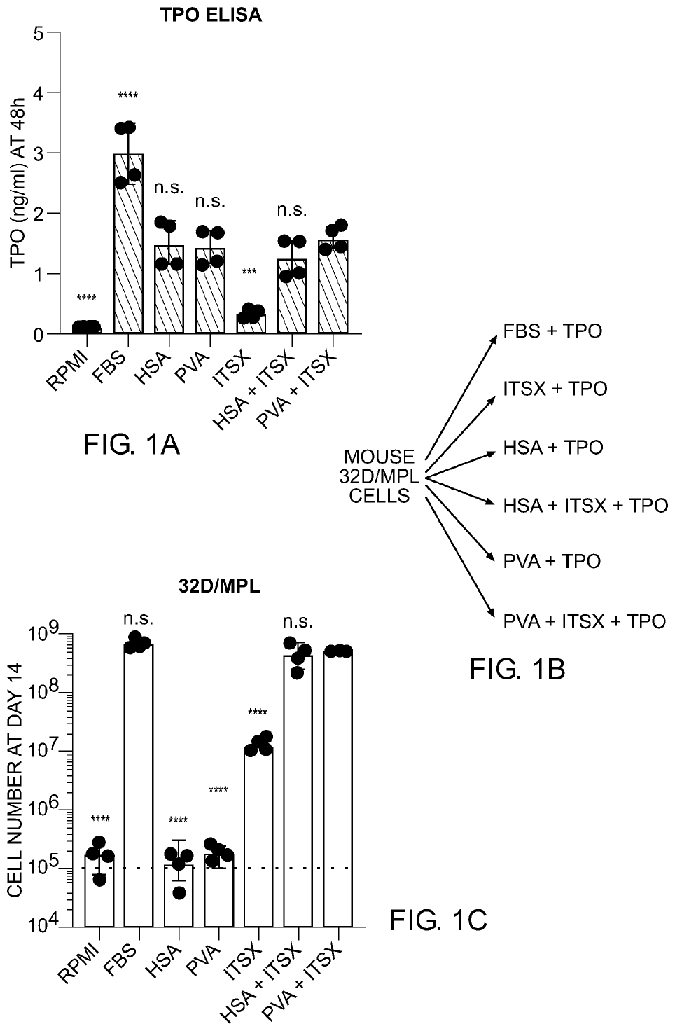

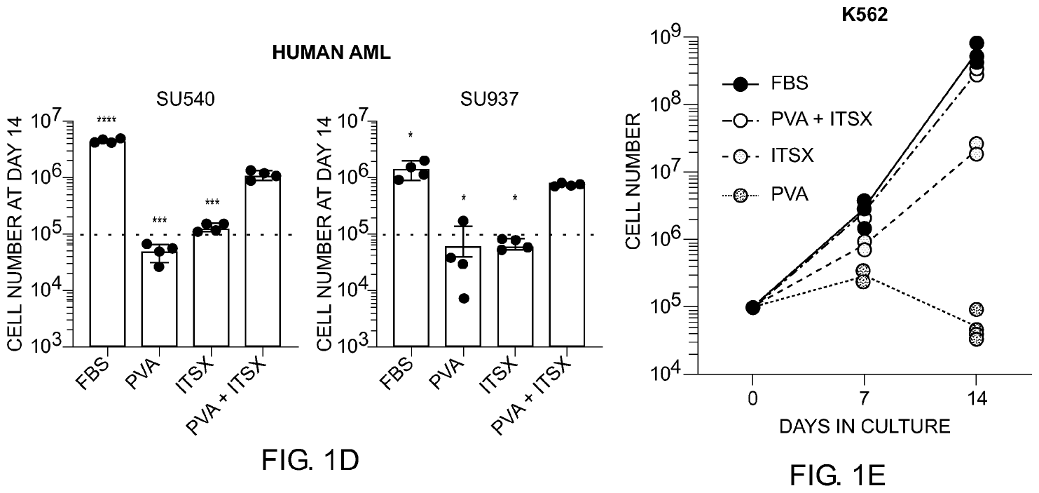

As a direct replacement for recombinant HSA in our HSC culture system, we hypothesized that PVA stabilizes proteins in the media, such as the key HSC cytokine thrombopoietin³ (TPO). Using ELISA assays, we confirmed that PVA stabilized TPO concentrations similar to HSA (FIG. 1A), while significantly more TPO was lost from the RPMI-only and ITSX-only medias (only ˜5% remained after 24 hours at 37° C. for ITSX-only). Consistent with our previous results with HSC media³ and the relative stability of TPO in these different media conditions, the proliferation of the thrombopoietin (TPO)-dependent 32D/MPL mouse cell line⁸ was highest in PVA+ITSX or HSA+ITSX conditions, comparable to expansion in FBS. By contrast, 32D/MPL cells more slowly proliferated in ITSX-only conditions, and essentially failed to grow in those conditions lacking insulin (FIGS. 1B, 1C).

To expand the applications of PVA in hematology research, we next tested the use of PVA in primary human acute myeloid leukemia (AML) cultures. PVA+ITSX supported growth of primary human AML samples cultured in the presence of cytokines, and almost equivalent to FBS containing media (FIG. 1D). PVA+ITSX also supported more rapid proliferation of the human erythroleukemia K562 cell line¹¹, without addition of exogenous cytokines (FIG. 1E). PVA may therefore have broad application for growing various hematological cell types beyond HSC cultures, although future work will need to determine how each cytokine/growth factor functions in albumin-free conditions. The PVA culture system also provides a useful platform with which dissect the biological components in serum that influence cell growth.

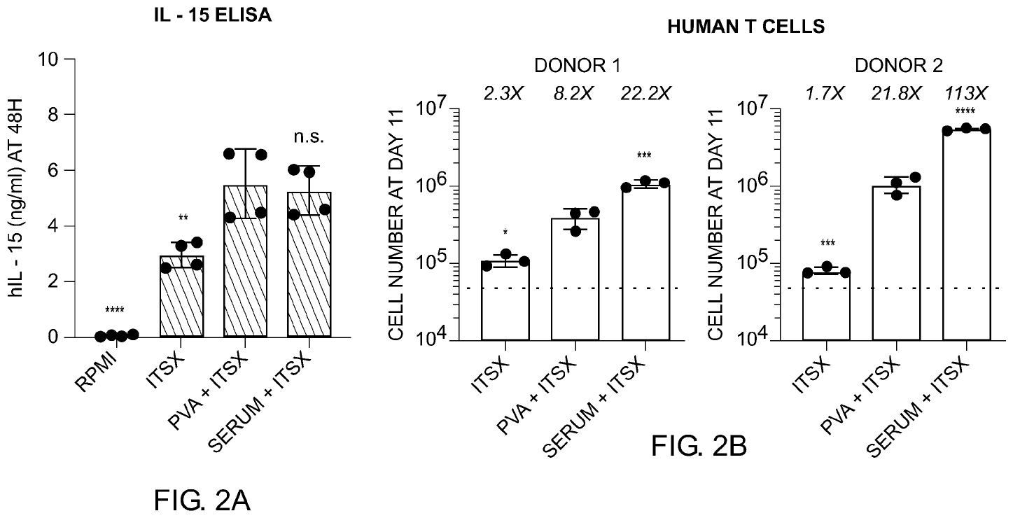

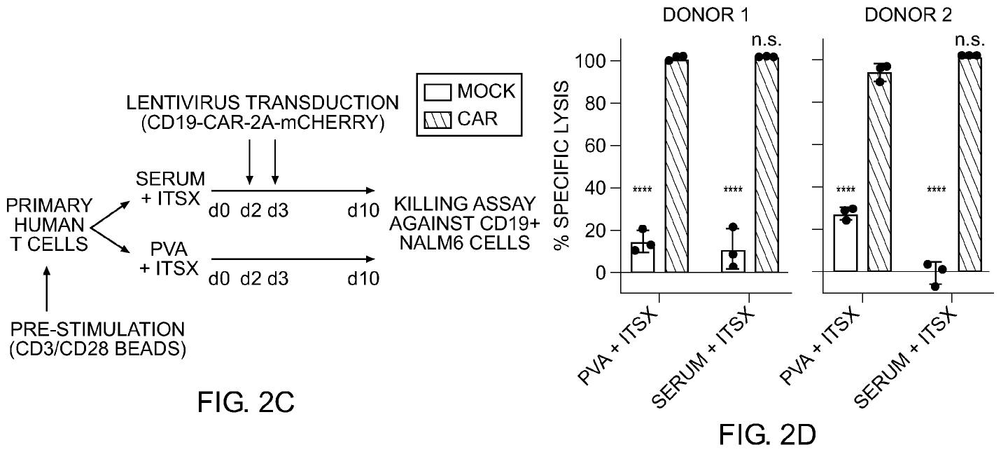

We also investigated the application of PVA to lymphocyte cell culture. We initially confirmed that the key T cell cytokine IL-15¹² was similarly stabilized in PVA+ITSX and Serum+ITSX medias (FIG. 2A). Primary human T cells expanded in serum albumin-free conditions containing PVA+ITSX by ˜8-22-fold over 11 days in culture (FIG. 2B). This expansion was slightly lower than in the Serum+ITSX cultures (˜22-113-fold), suggesting human serum contains additional factors promoting T cell growth, which remain to be identified. Consistent with the reduced stability of IL-15 in ITSX-only media, this condition supported only ˜2-fold expansion of T cells over 11 days (FIG. 2B). These results confirm that PVA can be used as a human serum-replacement for expanding primary human T cells and identify an inexpensive and chemically-defined carrier for human T cell culture.

For CAR T therapies, T cells must not only expand ex vivo, but also function to kill target cancer cells. We therefore confirmed that PVA-expanded T cells retained full functionality by assessing the use of PVA for expanding anti-CD19 CAR T cells¹³ (FIG. 2C). PVA+ITSX exerted comparable anti-CD19 killing activity as those expanded in Serum+ITSX (FIG. 2D). The replacement of serum albumin with PVA may therefore provide significant improvements in terms of both cost and safety for ex vivo T cell cultures.

In summary, PVA offers inexpensive and chemically-defined culture conditions for a number of cell types in hematology and immunology. We expect that these serum albumin-free media conditions will have important implications for how we culture cells for both basic research and clinical cell therapies.

### REFERENCES

1. Francis G L. Albumin and mammalian cell culture: implications for biotechnology applications. Cytotechnology. 2010; 62(1):1-16.

2. leyasu A, Ishida R, Kimura T, et al. An All-Recombinant Protein-Based Culture System Specifically Identifies Hematopoietic Stem Cell Maintenance Factors. Stem Cell Reports. 2017; 8(3):500-508.

3. Wilkinson A C, Ishida R, Kikuchi M, et al. Long-term ex vivo hematopoietic-stem-cell expansion allows nonconditioned transplantation. Nature. 2019.

4. June CH, O'Connor R S, Kawalekar O U, Ghassemi S, Milone M C. CAR T cell immunotherapy for human cancer. Science. 2018; 359(6382):1361-1365.

5. Ghorashian S, Amrolia P, Veys P. Open access? Widening access to chimeric antigen receptor (CAR) therapy for ALL. Exp Hematol. 2018; 66:5-16.

6. Ando M, Nakauchi H. ‘Off-the-shelf’ immunotherapy with iPSC-derived rejuvenated cytotoxic T lymphocytes. Exp Hematol. 2017; 47:2-12.

7. Levine B L, Miskin J, Wonnacott K, Keir C. Global Manufacturing of CAR T Cell Therapy. Mol Ther Methods Clin Dev. 2017; 4:92-101.

8. Seita J, Ema H, Ooehara J, et al. Lnk negatively regulates self-renewal of hematopoietic stem cells by modifying thrombopoietin-mediated signal transduction. Proc Natl Acad Sci USA. 2007; 104(7):2349-2354.

9. Bak R O, Dever D P, Reinisch A, Cruz Hernandez D, Majeti R, Porteus M H. Multiplexed genetic engineering of human hematopoietic stem and progenitor cells using CRISPR/Cas9 and AAV6. Elife. 2017; 6.

10. Fu X, Tao L, Rivera A, et al. A simple and sensitive method for measuring tumor-specific T cell cytotoxicity. PloS one. 2010; 5(7):e11867.

11. Lozzio B B, Lozzio C B, Bamberger E G, Feliu A S. A multipotential leukemia cell line (K-562) of human origin. Proc Soc Exp Biol Med. 1981; 166(4):546-550.

12. Klebanoff C A, Finkelstein S E, Surman D R, et al. IL-15 enhances the in vivo antitumor activity of tumor-reactive CD8+ T cells. Proceedings of the National Academy of Sciences of the United States of America. 2004; 101(7):1969-1974.

13. Milone M C, Fish J D, Carpenito C, et al. Chimeric receptors containing CD137 signal transduction domains mediate enhanced survival of T cells and increased antileukemic efficacy in vivo. Mol Ther. 2009; 17(8):1453-1464.

1. A method of culturing immune cells, the method comprising culturing the immune cells in a serum albumin-free medium comprising polyvinyl alcohol (PVA).

2. The method of claim 1, wherein the immune cells are lymphocytes.

3. The method of claim 2, wherein the lymphocytes are T cells.

4. The method of claim 3, wherein the T cells are chimeric antigen receptor (CAR) T cells.

5. The method of claim 1, wherein the serum albumin-free medium further comprises one or more cytokines or growth factors.

6. The method of claim 5, wherein the one or more cytokines or growth factors are selected from the group consisting of interleukin 1 (IL-1), interleukin 2 (IL-2), interleukin 3 (IL-3), interleukin 4 (IL-4), interleukin 6 (IL-6), interleukin 7 (IL-7), interleukin 9 (IL-9), interleukin 10 (IL-10), interleukin 10 (IL-12), interleukin 13 (IL-13), interleukin 15 (IL-15), interleukin 18 (IL-18), interleukin 21 (IL-21), interleukin 23 (IL-23), tumor necrosis factor alpha (TNFα), tumor necrosis factor beta (TNFβ), interferon gamma (IFN-γ), macrophage colony-stimulating factor (M-CSF), granulocyte-macrophage colony-stimulating factor (GM-CSF), thrombopoietin (TPO), stem cell factor (SCF), and FMS-like tyrosine kinase 3 ligand (FLT3L).

7. The method of claim 1, wherein the serum albumin-free medium further comprises an antibiotic.

8. The method of claim 7, wherein the antibiotic is selected from the group consisting of penicillin, streptomycin, penicillin-streptomycin, gentamicin, and amphotericin.

9. The method of claim 1, wherein the serum albumin-free medium further comprises insulin, transferrin, selenium, or ethanolamine, or a combination thereof.

10. The method of claim 1, wherein the PVA is at a concentration of about 0.1 mg/ml to about 10 mg/ml in the serum albumin-free medium.

11. The method of claim 10, wherein the PVA is at a concentration of about 1 mg/ml in the serum albumin-free medium.

12. The method of claim 1, wherein culturing is performed in a suspension culture or an adherent culture.

13. A composition comprising expanded immune cells cultured by the method of claim 1.

14. The composition of claim 13, further comprising a pharmaceutically acceptable excipient.

15. A method of cellular therapy comprising administering the composition of claim to a subject.

16. The method of claim 15, wherein the immune cells are autologous or allogeneic.

17. A method of adoptive cellular therapy with immune cells, the method comprising:
a) obtaining a biological sample comprising immune cells from a subject;
b) isolating the immune cells of interest from the biological sample;
c) expanding the immune cells in a serum albumin-free cell culture medium comprising polyvinyl alcohol; and
d) administering a therapeutically effective amount of the expanded immune cells of interest to the subject.

18. The method of claim 17, wherein the biological sample is blood.

19. The method of claim 17, wherein the immune cells are lymphocytes.

20. The method of claim 19, wherein the lymphocytes are T cells.

21. The method of claim 20, further comprising genetically modifying the T cells to produce a chimeric antigen receptor (CAR) prior to administering said T cells to the subject.

22. The method of claim 21, wherein the CAR specifically binds to a cancer epitope.

23-31. (canceled)
